(19) United States
(12) Patent Application Publication
(10) Pub. No.: US 2026/0340502 A1
(43) Pub. Date: Oct. 29, 2026

(54) SYSTEMS AND METHODS FOR GENERATING A RISK PREDICTION MODEL FOR MULTILEVEL POST-OPERATIVE COMPLICATIONS

(71) Applicant: NERv Technology Inc., Kitchener (CA)

(72) Inventors: William Michael Kovacs Pitman, Kitchener (CA); Raqibul Hayder Chowdhury, Kitchener (CA); Finnegan O'Hara, Kitchener (CA); Alexander Fricke, Kitchener (CA); Khaled Berry, Kitchener (CA)

(21) Appl. No.: 19/554,435

(22) Filed: Mar. 2, 2026

Related U.S. Application Data
(60) Provisional application No. 63/764,894, filed on Feb. 28, 2025.

(51) Int. Cl. G06N 20/00; G16H 15/00; G16H 50/30

(52) U.S. Cl. CPC G06N 20/00; G16H 15/00; G16H 50/30

(57) ABSTRACT

A system and method for predicting surgical complications, wherein the method comprises receiving a plurality of clinical factors associated with one or more patients; determining a first set of surgical complication predictions from the plurality of clinical factors and a second set of surgical complication predictions from the plurality of clinical factors, wherein the first set comprises one or more overall surgical complication predictions and the second set comprises one or more surgical complication predictions based on different classification levels; determining one or more aggregated surgical complication predictions based on the first set of surgical complication predictions and the second set of surgical complication predictions; and outputting a clinical summary based on at least the first set of surgical complication predictions, the second set of surgical complication predictions, and the one or more aggregated surgical complication predictions.

### CROSS REFERENCE TO RELATED APPLICATIONS

This application claims priority from U.S. Provisional Patent Application No. 63/764,894, filed Feb. 28, 2025, the disclosure of which is hereby incorporated in its entirety by reference.

### FIELD OF THE DISCLOSURE

The present disclosure relates to predicting post-operative complications, in particular, systems and methods for generating a risk prediction model for post-operative complications.

### BACKGROUND

Depending on a patient's medical condition, healthcare providers may recommend performing surgical procedures to improve the patient's quality of life, especially if the condition has already progressed beyond the effectiveness of non-invasive treatments. Surgical procedures may range from being minimally invasive to more complex approaches such as open surgery to treat different pathological conditions and restore body functions. Although modern healthcare has made advancements in developing more effective surgical techniques to improve patient outcomes, there still lies an inherent risk of post-operative complications including, but not limited to, hemorrhages, infections, leakages, ischemia, and sepsis.

One of the most dangerous complications of surgery is a complication known as anastomotic leakage. Anastomotic leakage may develop after an anastomosis is performed where two organs are surgically connected and is most commonly observed in gastrointestinal surgery. Anastomotic leakage leads to luminal contents leaking into the peritoneal cavity which may cause a cascade of deadly complications to arise. This typically involves a form of severe sepsis, peritonitis, morbidity, and it may lead to mortality.

Using traditional techniques, it can take three to seven days on average for an anastomotic leak to be diagnosed since healthcare providers typically wait for symptoms such as abdominal pain, fever, and tachycardia to arise. This is very dangerous, especially considering that every hour of delay causes a considerable increase in the morbidity and mortality risk for the patient.

Leak incidence rate from surgical procedures can vary from 1% to 40% in some cases. Causes behind the development of anastomotic leaks are still being studied with no definitive causes identified yet. There are however risk factors that are associated with high incidence rate such as, but not limited to, age, gender, organ tension, local ischemia, medical history, and surgical errors.

Physiological changes, such as tissue ischemia followed by necrosis, may occur before and during the development of an anastomotic leak. These changes may present themselves as subtle changes in the patient's vital parameters that may not be detected by the current standard of care.

Each year, 70 million major abdominal surgery (MAS) procedures are performed globally. MAS includes pancreatic, hepatobiliary, and colorectal surgeries with a primary anastomosis. These surgeries have a complication rate of 30-60%, 20% of which have significant detrimental effects that require invasive treatments and enhanced patient monitoring. The occurrence of such complications can have dire consequences, both acute and long-term, including a significant degree of morbidity and mortality for affected patients.

Given the impact of post-operative complications, it is important to ensure that patients are managed appropriately throughout the entire surgical process, from preoperative to postoperative care. Prior to completing surgical procedures, it is especially important to comprehensively evaluate the risks involved in light of possible complications that may arise thereafter. By doing so, healthcare providers can optimize and tailor their approaches early in the surgical process which can lead to improved patient outcomes.

This background information is provided to reveal information believed by the applicant to be of possible relevance. No admission is necessarily intended, nor should it be construed, that any of the preceding information constitutes prior art or forms part of the general common knowledge in the relevant art.

### BRIEF SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

The object of the present disclosure is a system and method for generating a risk prediction model for post-operative complications. Existing technologies are unspecific to the different multilevel classifications of post-operative complications, making them unsuitable in providing an accurate prediction. As such, there is a need for an optimized method of making predictions. Using data-driven techniques, the generated model provides a tool for healthcare providers to improve their decision-making when it comes to attending to surgical patients with utmost care.

In accordance with one aspect, there is provided a computer-implemented method for predicting surgical complications, the method comprising: receiving, from a server through a network, a plurality of clinical factors associated with one or more patients; determining, using a processor, a first set of surgical complication predictions from the plurality of clinical factors and a second set of surgical complication predictions from the plurality of clinical factors, wherein the first set comprises one or more overall surgical complication predictions and the second set comprises one or more surgical complication predictions based on a plurality of classification levels; determining, using the processor, one or more aggregated surgical complication predictions based on the first set of surgical complication predictions and the second set of surgical complication predictions; and outputting, using the processor, a clinical summary based on at least the first set of surgical complication predictions, the second set of surgical complication predictions, and the one or more aggregated surgical complication predictions.

In some embodiments, the plurality of clinical factors comprises types of procedures, medical history, current physiological conditions, demographics, intra-operative factors, laboratory test values, and qualitative assessment of health of the one or more patients.

In some embodiments, the one or more aggregated surgical complication predictions is determined a joint probability of a combination of one or more surgical combination predictions.

In some embodiments, the one or more aggregated surgical complication predictions is determined by a separate machine learning model that aggregates each of the one or more surgical combination predictions.

In some embodiments, the one or more aggregated surgical complication predictions is determined based on a threshold-based approach.

In some embodiments, the plurality of classification levels is based on severity of the one or more surgical complication predictions.

In some embodiments, the clinical summary further comprises intervention recommendations based on at least the first set of surgical complication predictions, the second set of surgical complication predictions, and the one or more aggregated surgical complication predictions.

In accordance with another aspect, there is provided a system for predicting surgical complications, the system comprising: a computing device connected to a server through a network, wherein the server comprises: a memory; and a processor coupled to the memory comprising program instructions, wherein the program instructions are executable by the processor to perform operations comprising: receiving a plurality of clinical factors associated with one or more patients; determining a first set of surgical complication predictions from the plurality of clinical factors and a second set of surgical complication predictions from the plurality of clinical factors, wherein the first set comprises one or more overall surgical complication predictions and the second set comprises one or more surgical complication predictions based on a plurality of classification levels; determining, using the processor, one or more aggregated surgical complication predictions based on the first set of surgical complication predictions and the second set of surgical complication predictions; and outputting a clinical summary based on at least the first set of surgical complication predictions, the second set of surgical complication predictions, and the one or more aggregated surgical complication predictions.

In some embodiments, the plurality of clinical factors comprises types of procedures, medical history, current physiological conditions, demographics, intra-operative factors, laboratory test values, and qualitative assessment of health of the one or more patients.

In some embodiments, the one or more aggregated surgical complication predictions is a joint probability of a combination of one or more surgical combination predictions.

In some embodiments, the one or more aggregated surgical complication predictions is determined by a separate machine learning model that aggregates each of the one or more surgical combination predictions.

In some embodiments, the one or more aggregated surgical complication predictions is determined based on a threshold-based approach.

In some embodiments, the plurality of classification levels is based on severity of the one or more surgical complication predictions.

In some embodiments, the clinical summary further comprises intervention recommendations based on at least the first set of surgical complication predictions, the second set of surgical complication predictions, and the one or more aggregated surgical complication predictions.

In accordance with another aspect, there is provided a non-transitory computer-readable medium storing program instructions, that when executed, cause one or more processors to perform operations comprising: receiving a plurality of clinical factors associated with one or more patients; determining a first set of surgical complication predictions from the plurality of clinical factors and a second set of surgical complication predictions from the plurality of clinical factors, wherein the first set comprises one or more overall surgical complication predictions and the second set comprises one or more surgical complication predictions based on a plurality of classification levels; determining, using the processor, one or more aggregated surgical complication predictions based on the first set of surgical complication predictions and the second set of surgical complication predictions; and outputting a clinical summary based on at least the first set of surgical complication predictions, the second set of surgical complication predictions, and the one or more aggregated surgical complication predictions.

In some embodiments, the plurality of clinical factors comprises types of procedures, medical history, current physiological conditions, demographics, intra-operative factors, laboratory test values, and qualitative assessment of health of the one or more patients.

In some embodiments, the one or more aggregated surgical complication predictions is a joint probability of a combination of one or more surgical combination predictions.

In some embodiments, the one or more aggregated surgical complication predictions is determined by a separate machine learning model that aggregates each of the one or more surgical combination predictions.

In some embodiments, the one or more aggregated surgical complication predictions is determined based on a threshold-based approach.

In some embodiments, the plurality of classification levels is based on severity of the one or more surgical complication predictions.

In some embodiments, the clinical summary further comprises intervention recommendations based on at least the first set of surgical complication predictions, the second set of surgical complication predictions, and the one or more aggregated surgical complication predictions.

Other aspects, features and/or advantages will become more apparent upon reading of the following non-restrictive description of specific embodiments thereof, given by way of example only with reference to the accompanying drawings.

### BRIEF DESCRIPTION OF THE DRAWINGS

Several embodiments of the present disclosure will be provided, by way of examples only, with reference to the appended drawings, wherein:

FIG. 1 is a flow chart illustrating an overview of an exemplary model of predicting post-operative complications, in accordance with one embodiment.

FIG. 2 is a flow chart illustrating a process of aggregating risk prediction values of post-operative complications as a joint probability, in accordance with one embodiment.

FIG. 3 is a flow chart illustrating a process of aggregating risk prediction values of post-operative complications by feeding the risk prediction values in a separate machine learning model, in accordance with one embodiment.

FIG. 4 is a flow chart illustrating a process of aggregating risk prediction values of post-operative complications using a threshold-based approach, in accordance with one embodiment.

FIG. 5 is a schematic diagram of a computing system used to determine post-operative complication predictions, in accordance with one embodiment.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

When introducing elements of aspects of the disclosure of the examples thereof, the articles “a,”, “an,”, “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

It is an object of the present disclosure to provide systems and methods for generating a risk prediction model for post-operative complications. Post-operative complications, such as anastomotic leakage, can be divided into different classifications based on certain criteria including, but not limited to, severity of the complication and type of recommended treatment pathway. One such classification is a standardized definition for anastomotic leaks established by the International Study Group of Rectal Cancer (ISREC) in 2010. The definition classifies anastomotic leaks into three different grades-grade A, grade B, and grade C-based on the impact on clinical management. Grade A anastomotic leakage results in no change in management of a patient that has no requirement of active therapeutic intervention. Grade B anastomotic leakage requires active therapeutic intervention and does not require reoperation such as a relaparotomy. Grade C anastomotic leakage requires reoperation. Based on the aforementioned definition, grade A anastomotic leakage may be categorized as a non-clinically relevant leak, while grade B and C anastomotic leakage may be categorized as clinically relevant leaks as these more significantly impact a patient's medical condition. Although the specification as described herein mainly discusses examples in relation to anastomotic leakage, the system and method described herein applies to other post-operative complications associated with grading systems such as ileus and post hepatectomy liver failure (PHLF).

The present disclosure provides a robust and efficient cascading model to predict post-operative complications, which specifically considers multilevel classifications of post-operative complications. This is in contrast to existing technologies which are restricted to generally considering a combination of different factors to predict a risk value associated with post-operative complications. For instance, high levels of amylase may indicate a risk of an anastomotic leak in a pancreatic surgery patient. However, this information is not directly indicative of whether the patient may have a grade A, grade B, or grade C anastomotic leak, and whether it is clinically relevant.

Given that different types of data are taken into account, the model is optimized to separate the task of predicting a general occurrence of a post-operative complication to predicting specifically a more advanced progression or version of the complication. Separating these tasks allows for machine learning algorithms to be optimized in their development to generate the risk prediction model for post-operative complications since different physiological factors and mechanisms may contribute towards the general occurrence of the overall complication and the more advanced progression or version of the complication. For example, surgical factors may cause the presence of an overall post-operative complication, but a patient's ability to heal may cause the complication to be more progressed, complex, advanced or impactful.

If the aforementioned tasks are not separated in the model, problems may likely arise in model optimization since the model may utilize clinical factors that contribute toward the general occurrence of a post-operative complication but does not have an impact on the more progressed version of the complication. In a similar manner, the model may utilize clinical factors that contribute to the more progressed version of a post-operative complication but does not have an impact on the general occurrence of the complication.

Another exemplary condition is provided herein in relation to the determination of surgical complication predictions. Overall anastomotic leak, regardless of its classification, may be determined based on laboratory tests that include amylase levels, as well as intraoperative factors such as pancreatic texture. However, clinically relevant anastomotic leak may be dependent on other factors comprising patient vitals, blood loss during surgery and/or a patient's ability to handle the anastomotic leak.

By generating an optimized machine learning model as described in the present disclosure, the prediction of post-operative complications provides a more reliable and accurate representation of the forecast of a patient's medical condition.

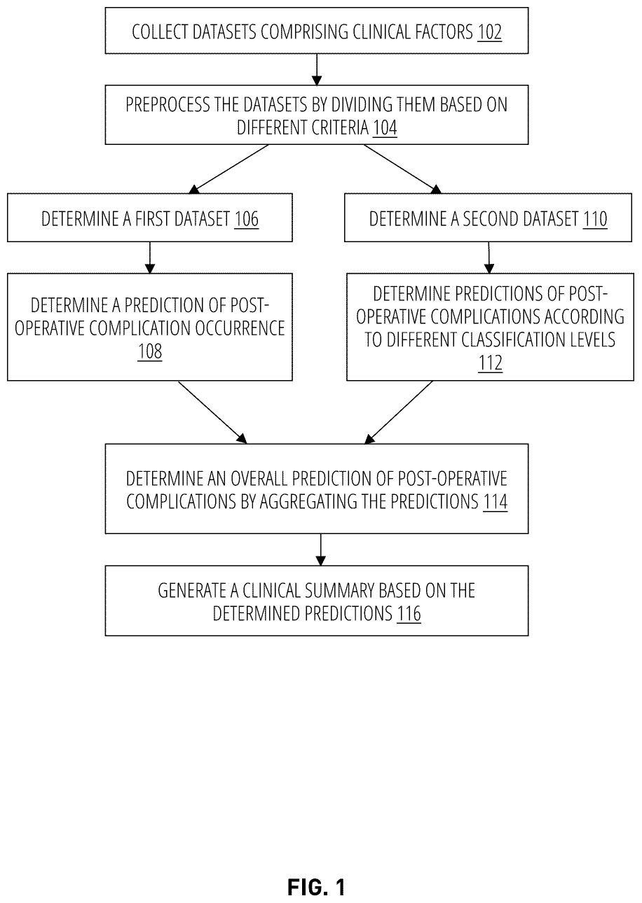

FIG. 1 illustrates an overview of an exemplary model of predicting post-operative complications. Datasets comprising a plurality of clinical factors associated with one or more patients are collected in step 102. The plurality of clinical factors may comprise types of procedures, medical history, current physiological conditions, demographics, intra-operative factors, laboratory test values, patient comorbidities, patient vitals, and qualitative assessment of health.

Laboratory test values may comprise different biomarker levels, wherein the term “biomarker” as used herein may refer to molecules, substances, and chemical or physical properties that can be measured or detected as bio-signals in bodily fluids. They include, but are not limited to, pH, temperature, electrolyte concentration, fluid flow rate, pressure, amylase, lactate, lactic acid, nitrates, alkali ions, inflammatory proteins, bacterial proteins, specific cells, molecules, genes, gene products, enzymes, hormones, inflammatory proteins, and glucose. Intraoperative factors may comprise blood loss and the condition of organs, wherein the condition of organs may comprise their texture, integrity, and presence of any infection, inflammation or injury.

Prior to determining predictions of post-operative complications, the collected datasets are preprocessed by dividing them based on different criteria in step 104, wherein a first dataset is determined in step 106 and second dataset is determined in step 110. The first dataset set may be based on at least laboratory tests, while the second dataset may be based on at least patient vitals. The determination of the different criteria is dependent on whether a given dataset significantly impacts the prediction of a more advanced progression or version of a complication. The model is optimized to separate the tasks of determining a general prediction of post-operative complications in step 108 based on the first dataset and determining predictions of post-operative complications according to different classification levels in step 112 based on the second dataset. In this embodiment, the different classification levels are based on the severity of post-operative complications. Once predictions have been determined based on the general occurrence and different classification levels, an overall prediction of post-operative complications is determined by aggregating the predictions in step 114. A clinical summary is then generated based on the aforementioned predictions in step 116 after determining the overall predictions by the separated tasks. The clinical summary further comprises intervention recommendations based on the predictions from the separate tasks, along with the aggregated predictions.

Different methods to aggregate or combine risk prediction values are described herein, resulting in an overall prediction of post-operative complications, such as anastomotic leaks. The determinants impacting the selection of an aggregation method may comprise an overall performance at clinically relevant predictions, alignment of clinical intuitions, and interpretability. An exemplary scenario is provided herein to demonstrate the selection process between the different methods to aggregate or combine risk prediction values. For instance, a user has an objective of optimizing the model to predict a clinically significant leak with the highest balanced accuracy. Each of the different methods of aggregation may be performed to determine the one that meets the aforementioned objective. However, having the method that yields the best performance may also come at a cost of requiring the use of a less interpretable non-linear model, making it unclear as to how exactly the scores are combined. In some embodiments, a user may view a score associated with each individual model along with an interpretable method of aggregating the scores such as the use of joint probability or decision thresholds as described herein. Having the user be informed of this information would allow for outputs of the model along the calculation path to be interpreted more effectively.

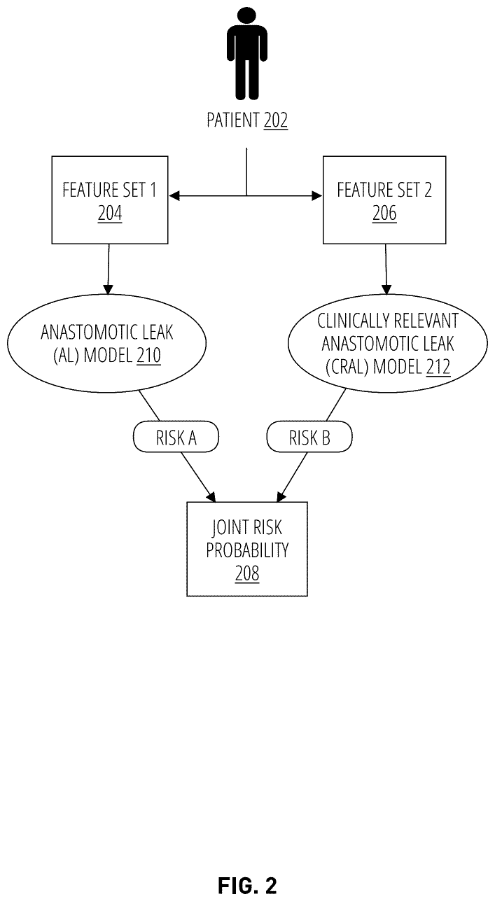

FIG. 2 illustrates a method of aggregating or combining risk prediction values of post-operative complications as a joint probability. In this case, the exemplary method illustrated is based on predictions of anastomotic leakage. With the datasets comprising a plurality of clinical factors associated with one or more patients 202, there lies a subset of data that is categorized further based on whether the data significantly impacts the clinical relevance of an anastomotic leak. Feature set 1 204 is a dataset relating to the mechanism of a general occurrence of an anastomotic leak, while feature set 2 206 is a dataset relating to the mechanism of a clinically relevant anastomotic leak. Feature set 2 206 can be further defined as covering a more advanced progression or version of post-operative complications. The features are optimized to each predictive task to produce probability predictions of each event.

As shown in the structure of the flow chart, the prediction of anastomotic leakage is separated wherein the prediction of the occurrence of any leak (grade A, B and/or C) is a distinct process to the prediction of a clinically relevant anastomotic leak (grade B and/or C). In a similar manner, but not illustrated, the prediction of any leak is a distinct process to the prediction of a non-clinically relevant anastomotic leak. An anastomotic leak (AL) model 210 generates a risk A value while a clinically relevant anastomotic leak (CrAL) model 212 generates a risk B value. Risk value A and B are aggregated based on probability theory to determine a joint risk probability 208. For example, the probability of a clinically relevant anastomotic leak (grade B and/or C) may be calculated using the following formula:

Pr(BC|leak)*Pr(leak).

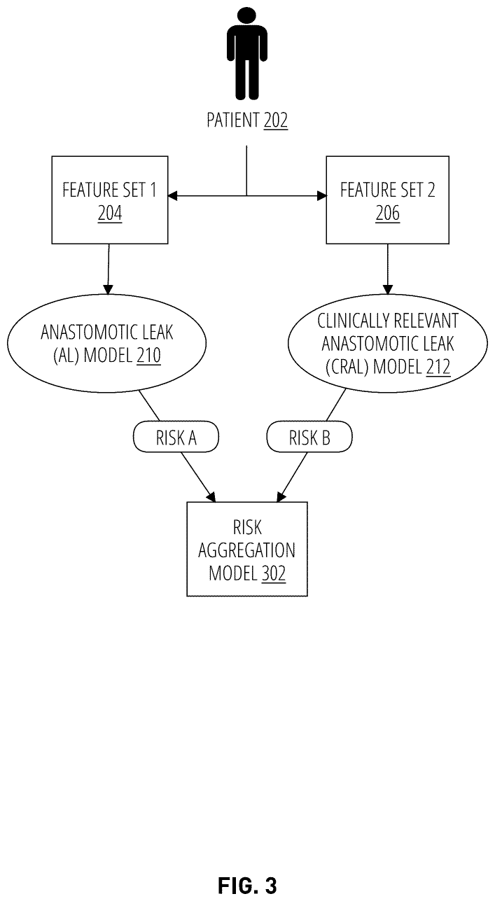

FIG. 3 illustrates a method of aggregating or combining risk prediction values of post-operative complications by feeding the risk prediction values in a separate machine learning model. In this case, the exemplary method illustrated is based on predictions of anastomotic leakage. When datasets comprising a plurality of clinical factors associated with one or more patients 202 are collected, the datasets are subjected to further refinement by creating subsets of data based on certain features such as whether the features contribute to the clinical relevance of an anastomotic leak. In the exemplary method shown, feature set 1 204 is a dataset relating to the mechanism of a general occurrence of an anastomotic leak, while feature set 2 206 is a dataset relating to the mechanism of a clinically relevant anastomotic leak. The features are optimized to each predictive task to produce probability predictions of each event.

As shown in the structure of the flow chart, the prediction of anastomotic leakage is separated wherein the prediction the occurrence of any leak (grade A, B and/or C) is a distinct process to the prediction of a clinically relevant anastomotic leak (grade B and/or C). In a similar manner, but not illustrated, the prediction of any leak is a distinct process to the prediction of a non-clinically relevant anastomotic leak. An AL model 210 generates a risk A value while a CrAL model 212 generates a risk B value. Risk value A and B are aggregated by feeding them into a separate risk aggregation model 302 that calculates an overall risk prediction. The separate risk aggregation model 302 may be trained to predict CrAL on an overall patient population based on the outputs of the AL model 210 and CrAL model 212. The separate risk aggregation model 302 is not the same as the AL model 210 and CrAL model 212 since each of the AL model 210 and CrAL model 212 are trained on different datasets. The AL model 210 is trained based on a general patient population with the label of whether or not they had an anastomotic leak, while the CrAL model 212 is trained specifically on a subset of the patient population that had experienced anastomotic leaks, wherein they are trained only with the label of whether or not they had clinically relevant anastomotic leak. In some embodiments, the separate risk aggregation model may use the information from each previous training task to isolate clinically relevant leaks from all other patients if that is the objective of the risk prediction model.

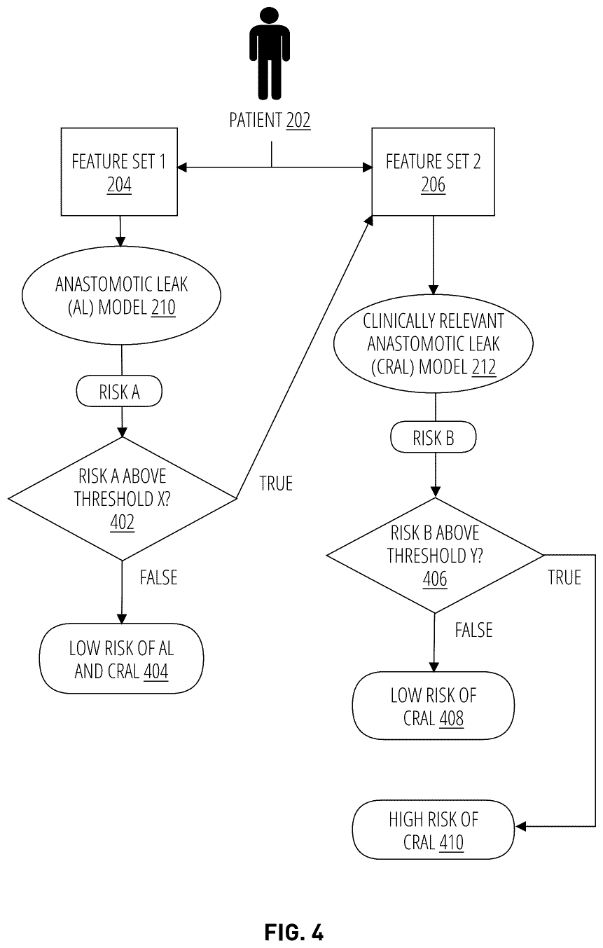

FIG. 4 illustrates a method of aggregating or combining risk prediction values of post-operative complications using a threshold-based approach. In the flow chart shown, the exemplary method illustrated is based on predictions of anastomotic leakage. When datasets comprising a plurality of clinical factors associated with one or more patients 202 are collected, the datasets are subjected to further refinement by creating subsets of data based on certain features such as whether the features contribute to the clinical relevance of an anastomotic leak. In the exemplary method shown, feature set 1 204 is a dataset relating to the mechanism of a general occurrence of an anastomotic leak, while feature set 2 206 is a dataset relating to the mechanism of a clinically relevant anastomotic leak. The features are optimized to each predictive task to produce probability predictions of each event.

As shown in the structure of the flow chart, the prediction of anastomotic leakage is separated wherein the prediction of the occurrence of any leak (grade A, B and/or C) is a distinct process to the prediction of a clinically relevant anastomotic leak (grade B and/or C). In a similar manner, but not illustrated, the prediction of any leak is a distinct process to the prediction of a non-clinically relevant anastomotic leak. An AL model 210 generates a risk A value while a CrAL model 212 generates a risk B value. Once the risk A and risk B values are determined, they are met with a decision point based on threshold X 402 and threshold Y 406, respectively. The thresholds are determined based on threshold values that provide an optimal overall performance when classifying a clinically relevant leak given certain constraints in relation to specificity, selectivity and accuracy, among others. The objectives for optimizing the overall risk prediction model may influence the set thresholds as well. For example, whether the objective is to generate less false positive results or less false negative results. Furthermore, the performance of each individual model may be another determinant of the thresholds. For example, each model may have its own receiver operating characteristic (ROC) curve with specific performance at its own classification task, which may influence the decision whether to choose one threshold over another.

In the AL model path, if risk A is not found to be above threshold X, it is determined that there is a low risk of an overall anastomotic leak 404, including a clinically relevant anastomotic leak. However, if risk A is found to be above threshold X, the risk values are fed into the CrAL model path. In the CrAL model path, if risk B is not found to be above threshold Y, it is determined that there is a low risk of a clinically relevant anastomotic leak 408. Otherwise, a high risk of a clinically relevant anastomotic leak is determined 410.

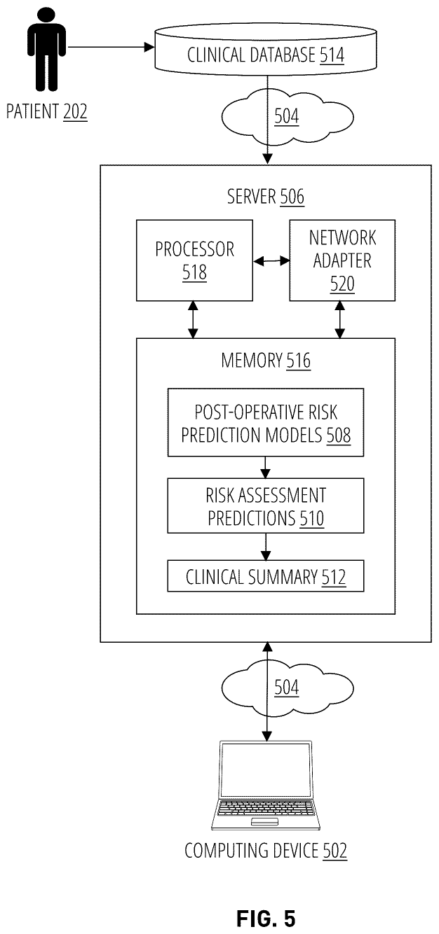

FIG. 5 is a schematic diagram of a computing system used to determine post-operative complication predictions. According to an embodiment of the present disclosure, the system includes datasets in a clinical database 514 based on information from one or more patients 202. The clinical database 514 is connected to a server 506 through a cloud computing network 504, wherein the connection between the clinical database 514 and the server 506 are facilitated by a network adapter 520. When a command is entered on a computing device 502 to determine risk prediction of post-operative complications for a given patient 202, the computing device 502 communicates with the server 506 through a network to execute program instructions stored in a non-transitory memory 516 using a processor 518. The non-transitory memory 516 is configured to store the post-operative risk prediction models 508, risk assessment predictions 510 and clinical summary 512. The program instructions within the non-transitory memory allow the processor to run the post-operative risk prediction models 508 to generate risk assessment predictions 510. When combined, the risk assessment predictions 510 serve as the basis of forming a clinical summary 512, which highlights different post-operative risks and may provide recommended interventions based on the severity of the predicted complications.

In some embodiments, the model is configured to predict complications 48 hours after surgery. Other embodiments of the model may be configured to predict complications at various post-operative time periods.

In some embodiments, methods other than those described above may be used to combine or aggregate predictions from the anastomotic leak model and the clinically relevant anastomotic leak model. For instance, thresholds from the prediction of each of the anastomotic leak model and the clinically relevant anastomotic leak model may be taken in conjunction with an overall prediction. An additional model may also fit the outputs of each model for the final prediction.

Depending on the classification of the prediction, the model may determine possible interventions to mitigate the predicted post-operative complications. For example, a non-clinically relevant anastomotic leak would not require a determination of possible interventions. However, a clinically relevant anastomotic leak may drive the model to provide suggested next steps such as a confirmatory CT scan and mitigation strategies to reduce its risk such as administering antibiotics, performing interventional radiology (IR) or reoperation. Based on the severity of a complication, the recommended interventions may be immediate or escalated as well.

Machine learning algorithms are applied to the collected datasets of a plurality of clinical factors associated with one or more patients. For example, an algorithm may recognize the patterns of the clinical factors categorized within an overall post-operative prediction path compared to a more narrowed category comprising either a clinically relevant or non-clinically relevant post-operative prediction model path. The machine learning algorithms generate a classification model trained by the previously acquired datasets to predict multilevel post-operative complications and distinguish it from predicting a general risk prediction of post-operative complications.

These algorithms may include, for example, Support Vector Machine (SVM), Naïve Bayes, Random Forest, Decision Trees, Logistic Regression, and Gradient Boosting Machines (GBM) such as XGBoost, NGBoost and LightGBM. In other embodiments, algorithms may also include, for example, deep learning architectures such as Deep Belief Network (DBN), Stacked Auto Encoder (SAE), Convolutional Neural Network (CNN) or Recurrent Neural Network (RNN). Other examples include, without limitation, Restricted Boltzmann machines (RBM), Social Restricted Boltzmann Machines (SRBM), Fuzzy Restricted Boltzmann Machines (FRBM), TTRBM models of Deep Belief Networks (DBN) or similar approaches could be used; AE, FAE, GAE, DAE, BAE models of Statistically Adjusted End Use (SAE) models could be used; models such as AlexNet, ResNet, Inception, VGG16, ECNN models of CNN may be used; Bidirectional Recurrent Neural Networks (BiRNN), Long Short-Term Memory (LSTM) networks, Gate Recurrent Unit (GRU) of RNN may also be used. Additional techniques specific to time-series modelling may be employed, including, but not limited to, dynamic time warping, change point detection, Autoregressive Integrated Moving Average (ARIMA). In some embodiments, other types of algorithms such as ordinal regression models may also be relied upon in conjunction with or in complementarity with those architectures and learning algorithms.

The present disclosure includes systems having processors to provide various functionality to process information, and to determine results based on inputs. Generally, the processing may be achieved with a combination of hardware and software elements. The hardware aspects may include combinations of operatively coupled hardware components including microprocessors, logical circuitry, communication/networking ports, digital filters, memory, or logical circuitry. The processors may be adapted to perform operations specified by a computer-executable code, which may be stored on a non-transitory computer readable medium.

The steps of the methods described herein may be achieved via an appropriate programmable processing device or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGAs), digital signal processors (DSPs), micro-controllers, and the like, programmed according to the teachings of the exemplary embodiments discussed above and appreciated by those skilled in the computer and software arts. Appropriate software can be readily prepared by programmers of ordinary skill based on the teachings of the exemplary embodiments, as is appreciated by those skilled in the software arts. In addition, the devices and subsystems of the exemplary embodiments can be implemented by the preparation of application-specific integrated circuits, as is appreciated by those skilled in the electrical arts. Thus, the exemplary embodiments are not limited to any specific combination of hardware circuitry and/or software.

Stored on any one or a combination of computer readable media or non-transitory computer readable media, the exemplary embodiments of the present invention may include software for controlling the devices and subsystems of the exemplary embodiments, for processing data and signals, for enabling the devices and subsystems of the exemplary embodiments to interact with a human user or the like. Such software can include, but is not limited to, device drivers, firmware, operating systems, development tools, applications software, and the like. Such computer-readable media further can include the computer program product of an embodiment of the present invention for preforming all or a portion (if processing is distributed) of the processing performed in implementations. Computer code devices of the exemplary embodiments of the present invention can include any suitable interpretable or executable code mechanism, including but not limited to scripts, interpretable programs, dynamic link libraries (DLLs), complete executable programs and the like.

Common forms of computer-readable media may include, for example, magnetic disks, flash memory, RAM, a PROM, an EPROM, a FLASH-EPROM, or any other suitable memory chip or medium from which a computer or processor can read.

Embodiments of the subject matter described in this specification can be implemented in a computing system that includes: a front end component (e.g., a computer having a graphical user interface or a Web browser though which a user can interact with an implementation of the subject matter described herein); or a midware component (e.g., an application server); or a back end component (e.g., a data server); or any combination of one or more such back end, middleware, or front end components. The components of the system can be interconnected by any form or medium of digital data communication (e.g., a communication network). Non-limiting examples of communication networks include a local area network (LAN) and a wide area network (WAN).

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Information as herein shown and described in detail is fully capable of attaining the above-described object of the present disclosure, the presently preferred embodiment of the present disclosure, and is, thus, representative of the subject matter which is broadly contemplated by the present disclosure.

What is claimed is:

1. A computer-implemented method for predicting surgical complications, the method comprising:
receiving, from a server through a network, a plurality of clinical factors associated with one or more patients;
determining, using a processor, a first set of surgical complication predictions from the plurality of clinical factors and a second set of surgical complication predictions from the plurality of clinical factors,
wherein the first set comprises one or more overall surgical complication predictions and the second set comprises one or more surgical complication predictions based on a plurality of classification levels of the surgical complications;
determining, using the processor, one or more aggregated surgical complication predictions based on the first set of surgical complication predictions and the second set of surgical complication predictions; and
outputting, using the processor, a clinical summary based on at least the first set of surgical complication predictions, the second set of surgical complication predictions, and the one or more aggregated surgical complication predictions.

2. The computer-implemented method of claim 1, wherein the plurality of clinical factors comprises types of procedures, medical history, current physiological conditions, demographics, intra-operative factors, laboratory test values, and qualitative assessment of health of the one or more patients.

3. The computer-implemented method of claim 1, wherein the one or more aggregated surgical complication predictions is a joint probability of a combination of one or more surgical combination predictions.

4. The computer-implemented method of claim 1, wherein the one or more aggregated surgical complication predictions is determined by a machine learning model that aggregates each of the one or more surgical combination predictions.

5. The computer-implemented method of claim 1, wherein the one or more aggregated surgical complication predictions is determined based on a threshold-based approach.

6. The computer-implemented method of claim 1, wherein the plurality of classification levels of the surgical complications is based on severity of the surgical complications.

7. The computer-implemented method of claim 1, wherein the clinical summary further comprises intervention recommendations based on at least the first set of surgical complication predictions, the second set of surgical complication predictions, and the one or more aggregated surgical complication predictions.

8. A system for predicting surgical complications, the system comprising:
a computing device connected to a server through a network, wherein the server comprises:
a memory; and
a processor coupled to the memory comprising program instructions, wherein the program instructions are executable by the processor to perform operations comprising:
receiving a plurality of clinical factors associated with one or more patients;
determining a first set of surgical complication predictions from the plurality of clinical factors and a second set of surgical complication predictions from the plurality of clinical factors,
wherein the first set comprises one or more overall surgical complication predictions and the second set comprises one or more surgical complication predictions based on a plurality of classification levels of the surgical complications;
determining, using the processor, one or more aggregated surgical complication predictions based on the first set of surgical complication predictions and the second set of surgical complication predictions; and
outputting a clinical summary based on at least the first set of surgical complication predictions, the second set of surgical complication predictions, and the one or more aggregated surgical complication predictions.

9. The system of claim 8, wherein the plurality of clinical factors comprises types of procedures, medical history, current physiological conditions, demographics, intra-operative factors, laboratory test values, and qualitative assessment of health of the one or more patients.

10. The system of claim 8, wherein the one or more aggregated surgical complication predictions is a joint probability of a combination of one or more surgical combination predictions.

11. The system of claim 8, wherein the one or more aggregated surgical complication predictions is determined by a machine learning model that aggregates each of the one or more surgical combination predictions.

12. The system of claim 8, wherein the one or more aggregated surgical complication predictions is determined based on a threshold-based approach.

13. The system of claim 8, wherein the plurality of classification levels of the surgical complications is based on severity of the surgical complications.

14. The system of claim 8, wherein the clinical summary further comprises intervention recommendations based on at least the first set of surgical complication predictions, the second set of surgical complication predictions, and the one or more aggregated surgical complication predictions.

15. A non-transitory computer-readable medium storing program instructions, that when executed, cause one or more processors to perform operations comprising:
receiving a plurality of clinical factors associated with one or more patients;
determining a first set of surgical complication predictions from the plurality of clinical factors and a second set of surgical complication predictions from the plurality of clinical factors,
wherein the first set comprises one or more overall surgical complication predictions and the second set comprises one or more surgical complication predictions based on a plurality of classification levels of the surgical complications;
determining, using the processor, one or more aggregated surgical complication predictions based on the first set of surgical complication predictions and the second set of surgical complication predictions; and
outputting a clinical summary based on at least the first set of surgical complication predictions, the second set of surgical complication predictions, and the one or more aggregated surgical complication predictions.

16. The non-transitory computer-readable medium of claim 15, wherein the plurality of clinical factors comprises types of procedures, medical history, current physiological conditions, demographics, intra-operative factors, laboratory test values, and qualitative assessment of health of the one or more patients.

17. The non-transitory computer-readable medium of claim 15, wherein the one or more aggregated surgical complication predictions is a joint probability of a combination of one or more surgical combination predictions.

18. The non-transitory computer-readable medium of claim 15, wherein the one or more aggregated surgical complication predictions is determined by a separate machine learning model that aggregates each of the one or more surgical combination predictions.

19. The non-transitory computer-readable medium of claim 15, wherein the one or more aggregated surgical complication predictions is determined based on a threshold-based approach.

20. The non-transitory computer-readable medium of claim 15, wherein the plurality of classification levels of surgical complications is based on severity of the surgical complications.

21. The non-transitory computer-readable medium of claim 15, wherein the clinical summary further comprises intervention recommendations based on at least the first set of surgical complication predictions, the second set of surgical complication predictions, and the one or more aggregated surgical complication predictions.
